(19) United States
(12) Patent Application Publication
(10) Pub. No.: US 2013/0273016 A1
(43) Pub. Date: Oct. 17, 2013

(54) FIBER AND PROBIOTICS FOR REDUCING INTESTINAL SYMPTOMS RELATED TO CHRONIC STRESS

(71) Applicants: Magali Faure, Forel (CH); Gabriela Bergonzelli Degonda, Bussigny (CH)

(72) Inventors: Magali Faure, Forel (CH); Gabriela Bergonzelli Degonda, Bussigny (CH)

(21) Appl. No.: 13/976,713

(22) PCT Filed: Dec. 28, 2011
(86) PCT No.: PCT/EP2011/074189

§ 371 Date: Jun. 27, 2013

(30) Foreign Application Priority Data
Dec. 29, 2010 (EP) 10197275.0
Mar. 1, 2011 (EP) 11156431.6

(51) Int. Cl. A61K 35/74

(52) U.S. Cl. CPC A61K 35/747
USPC 424/93.45; 424/780

(57) ABSTRACT

The present invention provides a composition that is suitable to reduce the impact of chronic stress on intestinal symptoms and/or conditions. The composition comprises a probiotic microorganism and soluble fiber. Preferably, the soluble fiber is a low viscosity soluble fiber. The composition is in particular suitable to alleviate abdominal discomfort, abdominal pain, abdominal cramps, and bowel movement disturbances; in as far as these conditions and symptoms are associated with stress.

The present invention relates to a composition, to the composition for reducing, treating and/or preventing stress-related intestinal symptoms and/or conditions, and to methods of relieving, treating and/or preventing stress-related intestinal symptoms and/or conditions. The present invention further relates to the composition in a method of relieving, treating and/or preventing intestinal symptoms and/or conditions, in particular symptoms and/or conditions associated with, promoted by and/or caused by stress.

### THE BACKGROUND ART AND PROBLEMS TO BE SOLVED BY THE INVENTION

An exposure to stressful situations is inescapable in our modern lifestyle. Stress can even become chronic, for example if stressful situations occur regularly or if a stressful situation is itself chronic. Daily stress has been implicated in the initiation and exacerbation of bowel and discomfort symptoms in IBS (Irritable Bowel Syndrome) patients (Whitehead W E, et. al. Effects of stressful life events on bowel symptoms: subjects with irritable bowel syndrome compared with subjects without bowel dysfunction. Gut. 1992; 33(6):825-30; Mayer E A. The neurobiology of stress and gastrointestinal disease. Gut. 2000; 47(6):861-9; and Blanchard E B et al. The role of stress in symptom exacerbation among IBS patients. Psychosom Res. 2008; 64(2):119-28).

Stress, be it chronic or acute stress, is also hypothesized to be partly responsible for, or to exacerbate gastrointestinal problems of the general population, but large correlation studies are lacking.

Exposure to stress, and in particular chronic stress, generally increases anxiety of individuals.

More than 50% of a representative sample of the general western population was recently shown to report gastrointestinal symptoms of discomfort, with flatulence, bloating and bowel movements irregularity being the most prevalent reported lower intestinal symptoms of discomfort (Van Kerkhoven L A et al. Gastrointestinal symptoms are still common in a general Western population. Neth J Med. 2008; 66(1):18-22). In Mexico, among 1025 subjects that answered the Rome II questionnaire, 377 (37%) of the subjects did not fulfil criteria for any functional gastrointestinal disorders, however they reported isolated symptoms such as hard/lumpy stools at least ¼ of the time during the previous 3 months, passing mucus (slime) during a bowel movement at least ¼ of the time during the previous 3 months, and abdominal fullness/bloating/swelling, at least ¼ of the time during the previous 3 months (Schmulson M et al. Rev. Gastroenterol. Méx. 2009; 74 (Supl. 2):23(33)).

In view of the above, it is an objective of the invention to elucidate the role of stress in the occurrence of intestinal conditions and symptoms.

It is an objective of the present invention to relieve gastrointestinal symptoms, in particular intestinal symptoms and most specifically lower intestinal symptoms.

It is an objective of the present invention to alleviate, treat and prevent abdominal discomfort, abdominal pain, abdominal cramps, and bowel movement disturbances.

It is an objective to regulate and/or restoring the regularity of bowel movements in subjects suffering from and/or at a risk of bowel movement irregularities.

It is an objective of the present invention to relieve, treat and prevent abdominal bloating, distension, flatulence, and constipation.

It is an objective of the present invention to relieve, treat and prevent diarrhea.

It is an objective of the present invention to reduce the frequency and the severity of the gastrointestinal conditions specified in this specification.

It is also an objective of the invention to reduce stress and anxiety of an individual, or the perception or subjective feeling of stress and anxiety, for example while being exposed to stress.

It is in particular an objective of the invention to reduce and alleviate the impact of stress on the symptoms and conditions specified in this specification, and to address the symptoms and conditions in as far as they are in any way related to, associated with, promoted by or even caused by stress in general, a stressful situation, acute stress, repeated exposure to stressful situations, and chronic stress.

It is an objective of the present invention to solve the above problems by way of nutrition, in particular by providing a nutritional composition suitable to relieve the above symptoms and conditions.

It is also an objective of the invention to provide nutrition or to improve the healthiness of food that an individual consumes. Ideally, the stress-related health symptoms and conditions are relieved, treated and/or prevented by the present invention in the absence of specific medication or any kind of medical treatment.

The present invention generally has the objective of improve overall well being of an individual, in particular the gastro-intestinal well-being.

Even more generally, the present invention has the objective to improve the quality of life of an individual.

The present invention addresses the problems and objectives set out above and elsewhere in this specification. The problems and objectives set out herein above are part of the present invention.

### SUMMARY OF THE INVENTION

Remarkably, the present inventors provide a new nutritional composition that relieves intestinal symptoms and in particular stress-related intestinal symptoms and/or conditions, which reduces the impact of stress on the intestinal symptoms, and which improves overall intestinal well-being.

Consequently, in a first aspect the present invention provides a composition comprising at least one probiotic and at least one soluble fiber.

In an aspect, the present invention provides a composition comprising at least one soluble fiber and at least one selected from (a) a live probiotic, (b) an inactive probiotic, (c) a culture medium of a probiotic and (d) a combination of two or more of (a), (b) and (c).

In a further aspect the invention provides a nutritional composition comprising at least one soluble fiber, at least one milk ingredient and at least one selected from (a) a live probiotic, (b) an inactive probiotic, (c) a culture medium of a probiotic and (d) a combination of two or more of (a), (b) and (c).

In an aspect the invention provides a nutritional composition comprising at least one soluble fiber, milk protein and at least one selected from (a) a live probiotic, (b) an inactive probiotic, (c) a culture medium of a probiotic and (d) a combination of two or more of (a), (b) and (c).

In a further aspect, the present invention provides a nutritional composition, comprising at least one source of available carbohydrates, at least one source of proteinogenic matter, and at least one soluble fiber, said composition further comprising at least one selected from (a) a live probiotic, (b) an inactive probiotic, (c) a culture medium of a probiotic and (d) a combination of two or more of (a), (b) and (c).

In an aspect, the present invention provides a composition comprising at least one soluble fiber, wherein fat provides about 20% or less, preferably 10% or less of the energy of the composition, and at least one selected from (a) a live probiotic, (b) an inactive probiotic, (c) a culture medium of a probiotic and (d) a combination of two or more of (a), (b) and (c). Therefore, the composition of the invention may but need not comprise one or more source of fat.

In a further aspect, the present invention provides the composition of the invention for relieving, treating and/or preventing intestinal conditions and/or symptoms.

In an aspect, the present invention provides a composition for regulating the digestive system of a subject.

In an aspect, the present invention provides the composition of the invention for improving the well-being, in particular intestinal well being.

In an aspect, the present invention provides the composition of the invention for relieving, treating and/or preventing one or more selected from abdominal discomfort, abdominal pain, abdominal cramps, and bowel movement disturbances and/or irregularities.

In an aspect, the present invention provides the composition of the invention for regulating bowel movements and/or restoring regular bowel movements.

In an aspect, the present invention provides the composition of the invention for regulating bowel movements in a subject suffering and/or at a risk of from bowel movement irregularities or disturbances, such as those specified in this specification.

In an aspect, the present invention provides the composition of the invention for restoring the regularity of bowel movements in a subject suffering from and/or at a risk of bowel movement irregularities or disturbances, such as those specified in this specification.

In an aspect, the present invention provides the composition of the invention for relieving, treating and/or preventing one or more selected from bloating, abdominal distension, flatulence, slow bowel transit, and constipation.

In an aspect, the present invention provides the composition of the invention for relieving, treating and/or preventing rapid or accelerated bowel transit and/or diarrhea.

In an aspect, the invention provides the composition of the invention for relaxing the digestive system, in particular of a subject.

In an aspect, the present invention provides the composition of the invention for relieving symptoms of digestive stress.

In a further aspect, the present invention provides the composition of the invention for reducing, relieving, treating and/or preventing stress and/or anxiety.

In an aspect, the present invention provides the composition of the invention for reducing, relieving, treating and/or preventing the perception and/or feeling of stress and/or anxiety of a subject.

In a further aspect, the present invention provides the composition of the invention for improving the quality of life.

In a further aspect, the present invention provides the composition of the invention for reducing stress-related intestinal conditions and/or symptoms.

In an aspect, the present invention provides the composition of the invention for reducing the impact of stress on intestinal conditions and/or symptoms.

In an aspect, the present invention provides the composition of the invention for reducing intestinal conditions and/or symptoms related to chronic stress.

In an aspect, the present invention provides the composition of the invention for reducing intestinal conditions and/or symptoms related to acute stress.

In an aspect, the present invention thus provides a composition comprising at least one soluble fiber and at least one selected from (a) a live probiotic, (b) an inactive probiotic, (c) a culture medium of a probiotic and (d) a combination of two or more of (a), (b) and (c), for relieving, treating and/or preventing intestinal symptoms and/or conditions related to chronic stress.

In an aspect, the invention provides a composition comprising at least one soluble fiber and at least one selected from (a) a live probiotic, (b) an inactive probiotic, (c) a culture medium of a probiotic and (d) a combination of two or more of (a), (b) and (c), for restoring regular, normal and/or healthy bowel movement in a subject suffering and/or at a risk of from bowel movement irregularities related to chronic stress.

In an aspect, the invention provides a method for providing nutrition, the method comprising the step of administering the composition of the invention to a subject.

In an aspect, the invention provides a method for improving the quality of life of a subject, the method comprising the step of administering the composition of the invention to a subject.

In a further aspect, the present invention provides a method for relieving, treating and/or preventing gastrointestinal conditions and/or symptoms, the method comprising the step of administering the composition of the invention.

In an aspect, the present invention provides a method for relieving, treating and/or preventing one or more selected from abdominal discomfort, abdominal pain, abdominal cramps, and bowel movement disturbances and/or irregularities, the method comprising the step of administering the composition of the invention.

In an aspect, the present invention provides a method for regulating bowel movements and/or restoring regular bowel movements, the method comprising the step of administering the composition of the invention.

In an aspect, the present invention provides a method for regulating bowel movements in a subject suffering from and/or at a risk of bowel movement irregularities or disturbances, such as those specified in this specification, the method comprising the step of administering the composition of the invention.

In an aspect, the present invention provides a method for restoring the regularity of bowel movements in a subject suffering from and/or at a risk of bowel movement irregularities or disturbances, such as those specified in this specification, the method comprising the step of administering the composition of the invention.

In an aspect, the present invention provides a method for relieving, treating and/or preventing one or more selected from bloating, abdominal distension, flatulence, slow bowel transit, and constipation, the method comprising the step of administering the composition of the invention.

In an aspect, the present invention provides a method for relieving, treating and/or preventing rapid or accelerated bowel transit and/or diarrhea, the method comprising the step of administering the composition of the invention.

In an aspect, the invention provides a method for improving the quality of life, the method comprising the step of administering the composition of the invention.

In an aspect, the present invention provides a method for relieving, treating and/or preventing stress-related gastrointestinal symptoms and/or conditions, in particular those specified in this specification.

In an aspect, the present invention provides a method for reducing, relieving, treating and/or preventing the impact of stress on intestinal conditions and/or symptoms, the method comprising the step of administering the composition of the invention.

In an aspect, the present invention provides a method for relieving, treating and/or preventing intestinal symptoms and/or conditions related to chronic stress, the method comprising administering to a subject in need thereof an effective amount of a composition comprising at least one soluble fiber and at least one selected from (a) a live probiotic, (b) an inactive probiotic, (c) a culture medium of a probiotic and (d) a combination of two or more of (a), (b) and (c).

Further aspects and preferred embodiments of the invention are provided in the appended claims and are set out in more detail in the detailed description herein below.

### DETAILED DESCRIPTION OF THE INVENTION

The present invention relates to a composition. The composition is intended for oral administration and/or consumption. In this regard, the composition comprises and preferably consists of edible components and/or matter. The composition is thus preferably free of any toxic and/or unwholesome matter, which is not intended or suitable for oral administration to a human or animal.

Within the context of this specification the word “comprises” is taken to mean “includes, among other things”. It is not intended to be construed as “consists of only”.

The composition of the invention need not be but preferably is a nutritional composition. A “nutritional composition”, for the purpose of this specification, is a composition comprising at least one nutrient. Nutrients may be selected from macronutrients, such as proteinogenic matter, available carbohydrates and sources of fatty acids and/or from micronutrients, such as vitamins and trace elements and the like. Preferred nutrients of the nutritional composition are specified elsewhere in this specification.

A nutritional composition may be a food product intended for human consumption, for example, a beverage, a drink, a bar, a snack, an ice cream, a dairy product, for example a chilled or a shelf-stable dairy product, a drink, for example a milk-based drink, a confectionery product, a cereal product such as a breakfast cereal, a frozen product intended for consumption after heating in a micro-wave or an oven, a ready-to-eat product, a fast food or a nutritional formula. A “chilled product”, is preferably a product that is stored at 1-10° C., preferably 2-8° C. and most preferably 3-6° C. before consumption, in particular in the time between the end of manufacturing and consumption.

A nutritional formula encompasses any nutritionally complete or supplementary formulation. It may be a generally applicable nutritional formula, an infant or baby formula, a formula for elderly patients, for intensive care patients, or a specially adapted formula for patients suffering from a specific disease, for example. For example the nutritional formula may be adapted to patients suffering from nutrition-linked problems, such as IBS (Irritable Bowel Syndrome), IBD (Irritable Bowel Disease, including Ulcerative colitis and Crohn's disease, hyperglycemia, obesity, weight loss, diarrhea, constipation, phenylketonuria, hepatitis, acute or chronic renal failure, just to mention a few. Any nutritional formula may be reconstitutable, that is, present in a dried form, or ready to drink, in the form of liquid formulas, for example. The nutritional formula may be a low-fat formula and/or a formula that can be consumed during any kind of diet.

Alternatively, or in addition, the composition of the invention may be a pharmaceutical composition or a dietary supplement. It may be provided in the form of tablets, pills, capsules, for example gelatine capsules, effervescent tablets, and the like, for example.

Accordingly, the present invention provides the composition of the invention for use in therapeutic treatment, in particular as a medicament.

The composition of the invention comprises fiber, in particular dietary fiber, and most preferably soluble fiber. The expression “soluble fiber” encompasses and preferably refers to carbohydrates or their derivatives that are resistant to digestion and absorption in the human small intestine with complete or partial fermentation in the large intestine. The soluble fiber is preferably a prebiotic.

The composition of the present invention preferably comprises 0.8 g or more of soluble fiber per serving. Preferably, the composition comprises 1 g or more, 1.2 g, 1.4 g, 1.6 g, 1.8 g, 2 g, and most preferably 2.1 g, 2.2 g, 2.5 g, 3 g, 4 g, 4.5 g, 5 g, 5.5 g, 6 g or more soluble fiber per serving. For example, the composition comprises between 1 g and 6 g of soluble fiber per serving. Preferred serving sizes of the entire composition of the invention are generally defined elsewhere in this specification. As an example, one serving can have about 23-25 g of dry matter.

Preferably, the daily served dose of soluble fiber by the composition of the invention is 1 g or more, preferably 2 g, 2.5 g, 3 g, 3.5 g, 3.8 g, 4 g, 4.1 g, 4.2 g, 4.3 g, 4.5 g, 4.7 g, 4.8 g, 5 g, 6 g, 7 g, 8 g, 9 g, 10 g, 11 g, 12 g, 13 g or more of soluble fiber. For example, the composition comprises between 3 g and 15 g of soluble fiber per daily served dose.

According to an embodiment, at least 5% by weight of dry matter of the composition is provided by said at least one soluble fiber. Preferably, the composition of the present invention comprises at least 5% 6%, 7%, 7.5%, 8%, 8.5%, 8.7%, 8.8%, 9%, 10%, 11%, 12%, 13%, 14% or more of soluble fiber in percent by weight of total dry matter of the composition.

The quantity of soluble fiber is preferably determined using the AOAC method: AOAC International. 1995. Total, soluble and insoluble dietary fiber in foods. AOAC official method 991.43. Official Methods of Analysis, 16^{th} ed.

The skilled person knows types of soluble fiber that are suitable for oral administration. Examples of soluble fiber are inulin, oligosaccharides such as fructo-oligosaccharides (FOS), xylooligosaccharides (XOS), galactooligosaccharides (GOS), mannooligo-saccharides, gluco-oligosaccharides, polydextrose, natural gums such as guar gum and acacia gum, mucilages, pectins, beta-glucans, tagatose, and resistant dextrins in general, besides resistant oligo-glucosaccharides.

According to an embodiment, the at least one soluble fiber comprises or substantially consists of soluble oligosaccharide fiber, such as fiber selected from FOS, XOS, GOS, mannooligo-saccharides, gluco-oligosaccharides and mixtures of two or more of the aforementioned.

The term “oligosaccharides”, for the purpose of the present specification, refers to carbohydrates comprising two or more identical or different monosaccharide units, which are connected by glycosidic bonds. “Oligosaccharides” include di-, tri-, tetra-, penta, hexa-, and so forth saccharides. Oligosaccharides may thus comprise 2 to 30, preferably 2-25, more preferably 2-20, even more preferably 2-17, 2-15, 2-13, 2-10, 3-9 monosaccharide units, which may be arranged in a linear or branched form in the oligosaccharide.

Generally, the oligosaccharide soluble fiber comprises a mixture of oligosaccharides of a different number of monosaccharide moieties (e.g. a mixture comprising di-, tri-, tetra- etc. -saccharides). According to an embodiment, the above indicated ranges (2 to 20, . . . , 3-9) thus preferably refers to the average number of monosaccharide moieties in the oligosaccharide fiber. The average is preferably the arithmetic mean based on the number of molecules.

Preferably, said at least one soluble fiber comprises or substantially consists of a low-viscosity soluble fiber. Preferably, “low-viscosity”, for the purpose of the present invention refers to a viscosity of below 5,000 cps (centipoises) of a 1% aqueous solution of the fiber when spun at 20 RPM with Brookfield RVT spindle #3, at a temperature of approximately 20-25° C. Preferably, “low viscosity” is a viscosity of 4,000 cps or lower, 3,000, 2,000, 1,000, 900, 800, 700, 600, 500, 400, 300, 200, 100, 90, 80, 70, 60, 50, 40, 30, 20, 15 cps or lower when tested at the above conditions.

According to an embodiment, the soluble fiber is selected from soluble fibers that have a viscosity of 3,000, 2,000, 1,000, 900, 800, 700, 600, 500, 400, 300, 200, 100, 90, 80, 70, 60, 50, 40, 30, 20, 15 cps or lower when tested in 2% aqueous solution of the fiber and spun at 20 RPM with Brookfield RVT spindle #3, at a temperature of approximately 20-25° C.

According to an embodiment, the soluble fiber is selected from soluble fibers that have a viscosity of 3,000, 2,000, 1,000, 900, 800, 700, 600, 500, 400, 300, 200, 100, 90, 80, 70, 60, 50, 40, 30, 20, 15 cps or lower when tested in 5%, preferably a 10% aqueous solution of the fiber when spun at 20 RPM with Brookfield RVT spindle #3, at a temperature of approximately 20-25° C.

According to a preferred embodiment, said at least one soluble fiber is or comprises resistant dextrin. Dextrins can be obtained from starch or glycogen using enzymes, such as amylases and/or by applying dry heat under acidic conditions (pyrolysis or roasting).

For the purpose of the present specification a “resistant” carbohydrate, for example a resistant dextrin, is a carbohydrate which cannot be digested by human digestive enzymes, but which may be metabolized by microorganisms present in the colon of a subject.

According to an embodiment, said at least one soluble fiber is provided by or comprises a resistant maltodextrin obtained from starch by a process comprising the steps of heat treatment (pyrolysis) and hydrolysis. Hydrolysis may be acid hydrolysis or enzymatic hydrolysis, but the latter is preferred.

Preferably, the resistant dextrin is a pyrodextrin.

A suitable process for obtaining resistant dextrins is described, for example, in U.S. Pat. No. 5,358,729 and U.S. Pat. No. 5,620,873.

Accordingly, the resistant dextrin may be obtained by preparing a slurry comprising water, starch and a mineral acid, and optionally mono- and/or oligosaccharides, if necessary, drying the slurry to a moisture content of 2 to 20%, preferably 3-10%, roasting the mixture at about 140-250° C., thereby obtaining a pyrodextrin.

The pyrodextrin is then preferably mixed with water and α-amylase, thereby obtaining a “pyro-maltodextrin”, and may be further processed to a powder.

Digestibility of the α-amylase treated pyrodextrin may be reduced by exposing, following treatment with α-amylase, the “pyro-maltodextrin” to a transglucosidase and/or to a (3-amylase (both, e.g. from Amano Enzyme USA). The two enzymes may be used alone or in combination.

The resistant dextrin obtained by such a process preferably contains about 50 wt.-%, 70 wt.-%, 80 wt.-%, 85 wt.-%, 90 wt.-%, 95 wt-% or more of fiber, in particular soluble fiber. “Wt.-%” is percent by weight of dry matter.

The resistant dextrin may also be referred to as resistant maltodextrin or simply maltodextrin.

The resistant maltodextrin is in particular a soluble oligo-glucosaccharide fiber.

According to an embodiment, the soluble fiber, in particular the resistant dextrin, comprises D-glucose moieties some of which are linked by α(1→2), and/or α(1→3) glycosidic bonds. These linkages are not found in natural starch. Because of the presence of several types of chemical bonds, including those above, the resistant dextrin is characterized by a much lower digestibility than natural starch or available dextrins. Resistant dextrin also contains α(1→4), and/or α(1→6) glucosidic bonds, which are present in natural starch. However, in the resistant dextrin, at least 4%, preferably at least 5%, 6%, 7%, 8%, 9%, 11%, 12%, 13%, 15%, of the glucose moieties of the resistant dextrin have an α(1→3) linkage. Preferably, at least 0.5%, 1%, 1.5%, 2% of the glucose moieties of the resistant dextrin have both, an α(1→2) and an α(1→4) linkage. Preferably, at least 5%, 6%, 7%, 8%, 9%, 11%, 12%, 13%, 15%, or more of the glucose moieties of the resistant dextrin have an α(1→6) linkage. The resistant dextrin preferably also contains levoglucosan.

Preferably, said resistant maltodextrin has a branched structure.

According to an embodiment, the resistant maltodextrin has a DE (dextrose equivalent) value of 2-30, preferably 5-20, even more preferably 6-15, for example 7-13, most preferably 8-12. According to an embodiment, the resistant maltodextrin (oligo-glucosaccharides have a DE of 3-14. According to an embodiment, said resistant dextrin is a resistant oligo-glucosaccharide.

According to an embodiment, the soluble fiber, for example the resistant dextrin, has an average molecular weight of 100-4500 Da, preferably 500-4000 Da, preferably 1000-3000 Da, more preferably 1500-2500 Da, most preferably 1800-2200 Da, for example about 2000 Da.

For example, the soluble fiber is a Fibersol® fiber, which is a commercially available resistant maltodextrin, in particular the soluble fiber is Fibersol-2® (www.fibersol2.com).

According to an embodiment, the composition of the invention comprises at least one selected from (a) a live probiotic microorganisms, (b) a non-replicating, for example, inactivated probiotic microorganisms, (c) the fermentation product of a probiotic microorganism, and (d) a mixture of two or more of the aforementioned (a), (b) and (c). According to an embodiment, the composition of the invention comprises at least one selected from (a) one or more live probiotic, (b) one or more inactive probiotic, (c) one or more culture medium of one or more probiotic and (d) a combination of two or more of (a), (b) and (c).

The composition of the invention thus comprises one or more probiotic and/or one or more medium fermented by one or more probiotic. A probiotic is a microorganism that is considered to be healthy for the host organism, for example a human or animal. In order to be able to exert a health benefit, the probiotic needs generally to be administered in adequate amounts. There are studies showing that the health benefit can also be conveyed by consumption of a medium that was fermented by a probiotic, even if the probiotic has been removed, and also by inactive probiotic. Without wishing to be bound by theory, it is hypothesized that metabolites produced by the probiotic may account, at least in part, for the health benefits provided by the probiotic, even if the microorganism is not replicating any more.

According to an embodiment, the term “probiotic”, for the purpose of the present specification, refers to any micro-organism that is able to exert the beneficial effects reported herein, or to a combination or mixture of such probiotics. A probiotic may thus be selected from known probiotic strains. However, a micro-organism so far not known to have probiotic properties may prove to have the beneficial effect according to the present invention and is therefore included within the term probiotic.

The expressions “probiotic”, “a probiotic”, “the probiotic”, and the like, for the purpose of the present specification, generally encompasses mixtures comprising different probiotics and probiotics in different form, for example mixtures comprising different probiotic species and/or strains. However, according to an embodiment, the expression “a probiotic” refers to a specific probiotic strain.

The literature mentions some of the micro-organisms from which the probiotics according to the present invention may be selected. For example, EP 0 862 863A2, in particular on page 3, lines 25-37, comprises a list from which the probiotic according to the present invention may be selected.

Examples of suitable probiotic micro-organisms include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Kocuria, Staphylococcus, Peptostrepococcus, Bacillus, Pediococcus, Micrococcus, Leuconostoc, Weissella, Aerococcus, Oenococcus and Lactobacillus.

Specific examples of suitable probiotic micro-organisms are: Aspergillus niger, A. oryzae, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, B. animalis, B. breve, B. bifidum, B. infantis, B. lactis, B. longum, B. pseudolongum, B. thermophilum, Candida pintolepesii, Clostridium butyricum, Enterococcus cremoris, E. diacetylactis, E. faecium, E. intermedius, E. lactis, E. muntdi, E. thermophilus, Escherichia coli, Kluyveromyces fragilis, Lactobacillus acidophilus, L. alimentarius, L. amylovorus, L. crispatus, L. brevis, L. casei, L. curvatus, L. cellobiosus, L. delbrueckii (for example: ss. bulgaricus), L. farciminis, L. fermentum, L. gasseri, L. helveticus, L. lactis, L. plantarum, L. johnsonii, L. reuteri, L. rhamnosus, L. sakei, L. salivarius, Leuconostoc mesenteroides, P. cereviseae (damnosus), Pediococcus acidilactici, P. pentosaceus, Propionibacterium freudenreichii, Prop. shermanii, Saccharomyces cereviseae, Staphylococcus carnosus, Staph. xylosus, Streptococcus infantarius, Strep. salivarius ss. thermophilus, Strep. thermophilus, Strep. lactis.

According to an embodiment, probiotic micro-organisms are selected from the group comprising or consisting of Bifidobacterium longum ATCC BAA-999; Bifidobacterium longum NCC 2705 (CNCM 1-2618, deposited on 29.01.2001); Bifidobacterium breve NCC 2950 (CNCM 1-3865, 15.11.2007); Bifidobacterium lactis NCC 2818 (CNCM I-3446, 07.06.2005); Lactobacillus johnsonii La1 (CNCM I-1225, 30.06.1992); Lactobacillus paracasei NCC 2461 (CNCM 1-2116, 12.01.1999); Lactobacillus rhamnosus NCC 4007 (CGMCC 1.3724, October 2004); Lactobacillus reuteri ATCC55730; Streptococcus thermophilus NCC 2019 (CNCM I-1422, 18.05.1994); Streptococcus thermophilus NCC 2059 (CNCM 1-4153, 24.04.2009); Lactobacillus casei NCC 4006 (CNCM 1-1518); Lactobacillus acidophilus NCC 3009 (ATCC 700396); Lactobacillus bulgaricus NCC 15 (CNCM 1-1198, 02.04.1992); Lactococcus lactis NCC 2287 (CNCM I-4154, on 24.04.2009); Lactobacillus paracasei ST11 (CNCM I-1292, 29.03.1993); and combinations thereof.

All these strains were either deposited under the Budapest treaty and/or are commercially available. The strains have been deposited under the Budapest treaty at the indicated deposit institution on the indicated date. Strains named CNCM were deposited with the COLLECTION NATIONALE DE CULTURES DE MICROORGANISMES (CNCM), 25 rue du Docteur Roux, F-75724 PARIS Cedex 15, France. Strains named CGMCC were deposited with the China General Microbiological Culture Collection Center, Institute of Microbiology, Chinese Academy of Sciences, Zhongguancun, P.O. Box 2714, Beijing 100080, China. Strains named ATCC are deposited at the American Type Culture Collection (www.lgcstandards-atcc.org).

According to an embodiment of the composition of the invention, said probiotic is a priobiotic strain selected from probiotics of the genera Bacillus, Bifidobacterium, Enterococcus, Saccharomyces, Lactobacillus, and Streptococcus.

According to a preferred embodiment, the probiotic is one or more selected from Lactobacillus paracasei, Lactobacillus fermentum and Lactobacillus delbrueckii. Preferably, said L. paracasei is or corresponds substantially to the strain deposited under deposit number CNCM I-2116.

It is noted that the life probiotic as well as the inactive probiotic may be, independently, be selected from the exemplary probiotic microorganisms disclosed in this specification or from other probiotic strains. Analogously, the medium fermented by a probiotic may be fermented, independently, by any one of the exemplary probiotic microorganisms or by probiotics that are available to the skilled person but are not disclosed herein.

The composition may comprise live probiotic and/or inactive, non-replicating probiotic. “Inactive probiotic” or “non replicating probiotic”, for the purpose of the present invention, is probiotic, which cannot multiply anymore, even under conditions that are normally conducive to the growth of the probiotic. According to an embodiment, inactive probiotic is dead probiotic, for examples the remains of killed probiotic. Inactive probiotic may be produced by exposing live probiotic to heat. In this case, the probiotic is heat-inactivated. If inactive probiotic is used in the composition of the invention, heat-inactivated probiotic is preferred.

The expression “one or more”, in particular in context of “one or more probiotic”, refers in particular to the possibility that only one probiotic strain is provided in the composition of the invention, and to the option that a mixture containing different probiotic strains is provided, or that different strains are separately provided in the composition of the invention.

According to an embodiment, the composition of the invention comprises a mixture of two or more, for example 2 to 10, 2 to 6, 2 to 5, 2 to 4, or 2 to 3 different probiotic strains. In said mixture, all strains may be alive, all strains may be heat-inactivated, and one or more strain may be heat-inactivated and one or more strain may be live.

In an embodiment, the invention encompasses a composition that is free of any live probiotic strain.

The invention also envisages a composition comprising only one strain, which is partly provided in live form and partly in inactivated form.

According to an embodiment, the composition of the invention comprises one or more live probiotic and/or on or more culture medium of one or more probiotic.

According to an embodiment, the composition of the invention comprises one or more inactive probiotic and/or one or more culture medium of one or more probiotic.

The invention thus encompasses compositions comprising one or more culture medium of one or more probiotic. A “culture medium” may be any substance of matter, for example a liquid or solid medium, in which at least one particular probiotic strain has grown for at least some time. The culture medium may be added in dried form (e.g. as a powder) or in liquid form to the composition of the invention. The at least one culture medium may, for example, comprise a dried or liquid concentrate of a medium that has at least partially been fermented by a probiotic. Preferably, the culture medium was supplied with and thus comprised nutrients at least before growing the probiotic, in particular nutrients that the probiotic was able to metabolize or grow on, such as a suitable source of carbohydrates. A “culture medium” may be a medium the nutrients of which may have been partially or completely metabolized and/or used up by the probiotic.

According to an embodiment, the composition comprises an ingredient based on a medium fermented by one or more probiotic, preferably a powder obtained from a medium fermented by one or more probiotic, wherein said medium comprises a milk ingredient. The milk ingredient may be selected from milk ingredients as defined elsewhere in this specification. For example, the medium may comprise skimmed milk or skimmed milk powder, milk protein and the like. According to an embodiment, the composition of the invention comprises an ingredient based on milk fermented by one or more probiotic, preferably a powder obtained from milk fermented by one or more probiotic.

For example, the composition may be produced using a medium fermented by a probiotic, said medium comprising one or more selected from milk, skimmed milk, milk protein (whey and/or casein), lactose, milk fat or a milk fat component. For example, the composition may be produced using yoghurt obtained by fermentation with a probiotic.

The medium fermented by a probiotic may be added in the form of a powder, for example a fermented milk powder, a powdered yoghurt, and the like, to the composition of the invention.

The probiotic may be provided in the form of a culture powder to the composition. Culture powders may be obtained, for example, by substantially removing the fermentation medium from the probiotic and spray drying the probiotic, possibly after adding suitable agents in order to increase dry matter content, such as carbohydrates and the like.

According to an embodiment, the composition of the invention comprises a mixture of two or more inactive probiotic strains, for example two or more heat-inactivated probiotics. According to a preferred embodiment, the mixture comprises some or all of the culture medium of one or more of the probiotic strains. The two or more probiotic strains may be cultivated separately, the different fermentation media may be mixed and dried together, or the different fermentation media may be dried separately and mixed thereafter, or the two or more probiotic strains may be grown in the same medium and dried together.

According to an embodiment, the composition of the invention comprises two or more live and/or inactivated lactobacilli strains. Preferably, the composition comprises a L. fermentum and a L. delbrueckii strain. Preferably, the composition comprises two or more inactivated lactobacilli strains. According to an embodiment, the composition comprises inactivated L. fermentum and/or inactivated L. delbrueckii, with or without fermented medium. For example, the composition comprises the commercially available product Lacteol®.

According to an embodiment, in the composition of the invention, the sum of all live probiotics, if present, is at least 10⁵, preferably at least 10⁶, 10⁷, 5×10⁷, 10⁸, 3×10⁸, 6×10⁸, 8×10⁸, 10⁹, 2×10⁹, 3×10⁹, 4×10⁹, 5×10⁹, 6×10⁹, 10¹⁰, 10¹¹, 10¹², or at least 10¹³ CFU per serving of the composition. Preferred serving sizes are disclosed elsewhere in this specification.

In this regard, as there is generally some loss of CFU during manufacturing and shelf-life of the composition, the indicated amounts preferably apply to the amount of live probiotic available when the product is consumed. Therefore, potential loss during manufacturing and shelf-life is preferably compensated by using/adding more live probiotics in the manufacturing process.

In case only inactive probiotics are present in the composition, the sum of all inactive probiotics, is at least 10⁵, preferably at least 10⁶, 10⁷, 5×10⁷, 10⁸, 3×10⁸, 6×10⁸, 8×10⁸, 10⁹, 2×10⁹, 3×10⁹, 4×10⁹, 5×10⁹, 6×10⁹, 10¹⁰, 10¹¹, 10¹², or at least 10¹³ CFU per serving, as determined before inactivating said probiotic.

According to an embodiment of the composition, the sum of all live probiotics, if present, is at least 5×10⁷, preferably at least 10⁸ CFU per 24 g of dry matter of the composition and/or wherein, in case only inactive probiotics are added, the sum of all inactive probiotics, is at least 5×10⁷, preferably at least 10⁸ CFU per 24 g of dry matter or per serving of the composition, as determined before inactivating said probiotic.

If mixtures of live and inactive probiotics are added to the composition, the indicated amounts preferably apply to the sum of both together, active and inactive CFU.

In general, the probiotic, be it active or inactive, and/or the medium fermented by the probiotic, is added in an amount that is sufficient to produce the beneficial effects reported herein. Without wishing to be bound by theory, it is believed that the above amounts in CFU are sufficient to produce the desired effects, along with the further components of the composition. In case fermented medium is added without any live probiotic, the amount of fermented medium is such that the advantageous effects reported herein are achieved.

Advantageously, thanks to the synergic combination of probiotic and/or its fermented medium in combination with soluble fiber in the composition of the invention, the amount of probiotic and/or fiber, each taken for itself, can be, but need not be, comparatively lower than if each had to be administered alone, without the respective other, in order to achieve the beneficial effects reported in this specification.

According to an embodiment, the composition of the invention comprises macro- and/or micronutrients.

According to an embodiment, the composition of the invention comprises at least one source of available carbohydrates.

The expressions “at least one” and “one or more” refer to one or more of the specific item referred to, for example, in the case of carbohydrates, to one or more types or sources of carbohydrates, for example, maltodextrin, starch, different types of starch (e.g. corn starch, potato starch, cassava starch, etc.), different types of maltodextrins, starch and maltodextrin, glycogen, sugars, and so forth. For the purpose of the present specification, in case there are more than one items present (for example: two sources of available carbohydrates), the expressions “the at least one”, “said at least one” and “said one or more”, etc., generally refer to all items (for example: both of the two sources of available carbohydrates). Accordingly, the expression “said at least one source of available carbohydrates provides 40% to 90% of the energy of the composition” is intended to mean that all sources of available carbohydrates present in the composition together provide the indicated amount of energy. The same applies, of course, to other nutrients or elements of the composition (such as possibly protein, fat, vitamins, trace elements), and to other characteristics than energy, such as weight, weight percentage, wt. % of dry matter, and so forth, for example.

“Available carbohydrates” represents that fraction of carbohydrate that can be digested by human enzymes, is absorbed and enters into intermediary metabolism. Available carbohydrates do not include dietary fibre.

Examples of available carbohydrates are starches, (available) maltodextrins, sugars, including monosaccharides, such as glucose, fructose, and galactose, disaccharides, such as saccharose (sucrose), lactose, maltose, trisaccharides and oligosaccharides, for example.

The composition of the invention may contain lactose. According to another embodiment, the composition of the invention is low in lactose or substantially lactose free. “Low in lactose” preferably means that less than 40%, preferably less than 30%, less than 20%, less than 10%, preferably less than 5%, most preferably less than 3% by weight of available carbohydrates being provided by lactose. In case there are no other available carbohydrates in the composition, “low in lactose” means that less that 10% by weight of the composition, preferably less than 5%, more preferably less than 3% and most preferably less than 2% by weight of the composition is lactose. “Substantially lactose free” means that lactose preferably provides less than 2%, preferably less than 1% and most preferably less than 0.5% of the weight of available carbohydrates of the composition. In case there are no other available carbohydrates than lactose in the composition, “substantially lactose free” means that less than 1%, preferably less than 0.5% most preferably less than 0.3% by weight of the composition are provided by lactose. Percent by weight are percent by weight of dry matter for the purpose of this specification, unless otherwise indicated.

Preferably, at least 10%, more preferably at least 20%, 30% per weight of the available carbohydrates of the composition are available maltodextrin. Preferably, at least 10%, more preferably at least 20%, 30% per weight of the available carbohydrates of the composition is lactose.

According to an embodiment, 10-97%, preferably 15-95% per weight of dry matter of the composition are available carbohydrates, preferably 40-80%, more preferably 50-70%, most preferably 55-65%.

For the purpose of the present specification, percentages per weight are percentage by weight of dry matter.

Preferably, available carbohydrates provide 10% to 100%, preferably 20% to 95%, more preferably 40% to 90%, preferably 55% to 85%, for example 60% to 80% of the energy of the composition. For example, available carbohydrates provide about 65% to about 75% of the energy of the composition.

According to an embodiment, the composition of the invention comprises at least one protein source and/or at least one source of proteinogenic matter. For the purpose of the present specification, the expressions “a source of protein”, “protein” and “a source of proteinogenic matter” in general encompasses any proteinogenic matter. These expressions thus encompass proteinogenic amino acids, dipeptides, oligopeptides, polypeptides, proteins, and the like, and mixtures of the aforementioned. The expression “source of protein” also encompasses protein hydrolysates, for example.

Protein sources may be of plant, fungal or animal origin, for example.

According to an embodiment, the nutritional composition comprises proteinogenic matter originating from milk protein. Preferably, the composition of the invention comprises milk protein. Preferably, the composition comprises casein and/or whey.

According to an embodiment, 4-50% per weight of dry matter of the composition are proteins (proteinogenic matter), preferably 10-35%, preferably 13-30%, more preferably 15-27%, most preferably 17-25%.

According to an embodiment, the at least one protein source (proteinogenic matter) provides 5% to 50% of the energy of the composition. Preferably, said at least one protein source provides 10% to 40%, more preferably 15% to 35%, even more preferably 18% to 32%, still more preferably 20% to 30% and most preferably 22% to 28% of the energy of the composition.

According to an embodiment of the composition of the invention, the at least one source of available carbohydrates provides 40% to 90%, preferably 55% to 85% of the energy of the composition, and said at least one protein source (proteinogenic matter) provides 10% to 40%, preferably 15% to 30% of the energy of the composition.

The composition of the invention may be free of fat and/or may not contain any source of fat. This applies independently to compositions that comprise lactose, are low in lactose or lactose free. However, according to an embodiment, the composition of the invention comprises at least one source of fat. The expression “fat”, for the purpose of the present specification, includes any oil or fat suitable for human consumption, in particular animal or vegetal oils and/or fats. According to an embodiment, the composition of the invention comprises milk fat.

According to an embodiment, the composition of the invention is a low-fat nutritional composition. Preferably, the nutritional composition comprises 10% by weight of dry matter or less fat, preferably 9%, 8%, 7%, 6%, 5%, 4%, 3%, 2% or less fat by weight of dry matter of the composition.

According to an embodiment, said optional and/or at least one fat source, if present in the composition, provides 40% or less, 30% or less, 25% or less, preferably 20% or less, more preferably 15%, 10%, 9%, 8%, 7%, 6%, 5% or less of the total available energy of the composition. Preferably, the optional one or more source of fat provides 0% to 25%, preferably 0.5% to 15%, most preferably 1% to 10% of the total energy of the composition.

Preferably, the composition of the invention comprises micronutrients. Preferably, the composition is supplemented with vitamins and or trace elements. Preferably, one serving/administration dose of the composition provides at least 15%, preferably at least 20%, 30%, 40% and most preferably at least 50% of the daily recommended allowances (RDAs) of one, two or more or all selected from vitamin A, vitamin D, vitamin E, vitamin C, vitamin B1, niacin, vitamin B6, folic acid, and biotin. Preferably, one serving/administration dose of the composition provides at least 20%, preferably at least 40% and most preferably at least 50% of the daily recommended allowances of iron.

RDAs of vitamins and trace elements, for the purpose of this specification, are as defined in the Commission Directive 2008/100/EC of 28 Oct. 2008.

Preferably, the composition comprises iodide, zinc, chromium, and molybdenum.

According to an embodiment, the composition of the invention comprises nucleotides, for example one or more nucleotides selected from uridine monophosphate (UMP), cytidin monophosphate (CMP), adenosine monophosphate (AMP), and guanosine (GMP). According to an embodiment, the composition comprises at least 5 mg, preferably at least 6 mg, 7 mg, 8 mg, 9 mg, and at least 10 mg of nucleotides per 100 g of dry matter of the composition.

According to an embodiment, the composition of the invention comprises at least one milk ingredient. A milk ingredient may be any fraction or any nutrient obtained from milk. The milk ingredient may, for example, be selected from lactose, milk protein, in particular casein and/or whey protein, milk fat, whole milk, skimmed milk, the milk fat globule membrane, whey in general, for example acid or sweet whey, or any particular whey protein in isolated form, for example one or more selected from β-lactoglobulin, α-lactalbumin, bovine serum albumin and immunoglobulins. The milk ingredient may be a mixture of one, two or more of the above mentioned. Any milk ingredient may, independently from other ingredients or other milk ingredients, be added in liquid form (if applicable) or may be added in a dried, in particular in a powdered form to the composition of the invention.

Milk and milk ingredients are advantageous as they provide valuable macro- and micronutrients. Milk contains, for example, substantial amounts of biotin, choline, inositol and L-carnitine, wherein the term “substantial amounts” refers to an amount that is suitable to cover a substantial percentage, for example, in case of biotin and choline, at least 3%, preferably at least 5%, 8%, 10%, 15%, 20% or more of the daily dietary reference intake of the respective nutrient of an individual, in particular in one serving of the composition of the invention (Dietary Reference Intakes (DRIs): Recommended Intakes for Individuals, Food and Nutrition Board, Institute of Medicine, National Academies, 2004, www.nap.edu). In this regard, the above percentages preferably apply to the DRI for a 19-30 year old female.

According to an embodiment, the said at least one milk ingredient of the composition of the invention comprises milk protein. The composition may comprise casein, whey or both. The casein and/or the whey protein may, independently, be provided in the form of a protein hydrolysate.

According to an embodiment, said at least one milk ingredient is based on skimmed milk.

For the purpose of the present specification, the expression “to be based on”, as found, for example, in the expression “to be based on skimmed milk” refers to the fact that the composition comprises an ingredient that corresponds to or is in any way obtained from the indicated raw material or ingredient, for example by further processing, in particular drying. For example, the expression “to be based on skimmed milk” includes the situation where the composition comprises skimmed milk or an ingredient obtained from skimmed milk, such as skimmed milk powder, for example.

As mentioned elsewhere in this specification, the composition may be provided in the form of a nutritional composition.

The composition of the present invention may be provided in liquid or in a dry form. In particular, the composition may be provided in the form of a powder that can be reconstituted by adding a liquid, such as milk, juice, water, preferably water, before consumption. If the composition is provided in liquid form, it may be shelf stable, preferably at room temperature (25° C.) and/or chilled, refrigerated, for example requiring refrigeration, or frozen, for example. If the composition is in liquid form, it is preferably ready-to-drink.

If the composition is provided in a liquid form, the probiotics or the fermentation medium, as applicable, may be present in the liquid but are preferably provided in a separate compartment in a dry form, so as to be combined with the liquid by the consumer shortly before consumption. This applies in particular if the probiotic is a live probiotic. In this way, undesired fermentation of the liquid composition by the probiotic before consumption is prevented and/or the survival of the probiotic up to consumption is improved or warranted.

A serving of the composition of the present invention, in case provided in the form of a nutritional composition, preferably has about 5 to 50 g dry matter, preferably 10-40 g, 15-35 g, more preferably 20-30 g and most preferably 21-27 g of dry matter, for example about 24 g dry matter.

The expression “serving”, and its various grammatical forms, for example in the expressions “a serving”, “serving size” or “serving per day”, and “served dose”, which terms are typically used in nutrition, also encompasses the expressions “administration”, “administration dose”, “administered dose”, “administered unit” and its grammatical forms. These latter expressions are more frequently used in the context of pharmaceutical compositions, which are also encompassed by the present invention. A “serving” “has its typical meaning as used in the art, and/or represents the amount of the composition that is administered at one moment of intake, for example as one meal or before, during and/or after a meal, before going to bed and so forth.

According to an embodiment, the nutritional composition of the invention is provided in a powdered, reconstitutable form.

If the composition of the invention is a powdered, reconstitutable nutritional composition, one serving size of powdered composition is preferably mixed with about 50-500 ml, 100-400 ml, preferably 100-300 ml, more preferably 150-250 ml, for example about 200 ml water.

If the composition is provided in the form of a liquid, ready-to-drink nutritional composition, one serving size corresponds to the amounts and preferred amounts of liquid and dry matter indicated above.

It is noted that in case the nutritional composition is consumed in liquid form, the fact that the soluble fiber is substantially provided in the form of a low-viscosity soluble fiber as mentioned above is particularly advantageous, as it provides a readily and easily drinkable, low-viscosity drink. Alternatively, if the nutritional composition is provided in a higher-viscosity or non-liquid form, for example in the form of a set or stirred yoghurt, the occurrence of soluble fiber providing higher viscosity to the composition is not necessarily a disadvantage.

According to an embodiment, the daily serving of the composition of the invention encompasses preferably two servings per day. In this way, it is possible to determine from the indications given above and elsewhere in this specification, referring to a serving, the preferred daily administered or served amount of any particular component or ingredient of the composition, by simple multiplication with 2. Of course, although two servings per day are preferred, it is possible to administer the daily administered amount in three or more servings, or in only one serving, for example. In this case, the size of the serving is preferably adjusted accordingly. Furthermore, the amount of the composition that needs to be administered in order to achieve the beneficial effects reported in this specification may be adjusted in dependence of the particular subject who wishes to enjoy the beneficial effects reported in this specification.

Preferably, one serving of the composition is consumed in the morning, and one serving in the afternoon, the evening or before going to bed. Preferably, one serving is taken at breakfast and one serving is administered at dinner or after dinner, before going to bed, for example.

According to an embodiment, one serving of the composition of the invention is part of or forms the breakfast of a human subject. This applies in particular to subjects that do not normally consume a consistent breakfast in the morning, or who do not take any breakfast (coffee or tea not being counted as a “breakfast”).

For example preferred daily serving of probiotics, can be determined, from the indications with respect to the preferred amount of probiotic administered per serving, as disclosed elsewhere in this specification, to be preferably at least 2×10⁵, preferably at least 2×10⁶, 2×10⁷, 10×10⁷ (10⁸), 2×10⁸, 6×10⁸, 12×10⁸ (1.2×10⁹), 16×10⁸ (1.6×10⁹), 2×10⁹, 4×10⁹, 2×10⁹, 6×10⁹, 8×10⁹, 10×10⁹ (10¹⁰), 12×10⁹ (1.2×10¹⁰), 2×10¹⁰, 2×10¹¹, 2×10¹², or at least 2×10¹² CFU per daily administered dose of the composition. The amounts preferably apply to the sum of all probiotics providing the benefits reported herein, for example live probiotics.

Similarly, the amount of a daily serving of soluble fiber may be comprehended from indications with respect to the amount of soluble fiber per serving specified elsewhere in this specification, assuming that two servings are consumed per day.

Similarly, the preferred weight, weight percentage or weight percent of dry matter of any component or ingredient of the composition may be determined on the basis of indications of an amount of said component or ingredient per serving size, and/or daily serving, and vice versa, on the basis of the indications in this specification.

The composition of the present invention provides several benefits, in particular health benefits. The composition also improves the quality of life of a subject.

According to an embodiment, the composition of the invention is suitable to reduce, treat and/or prevent stress, such as, for example chronic stress and/or acute stress.

According to a definition, the term “stress” refers to or encompasses psychological and physical reactions of a living being, which are caused by specific external stimuli. Said psychological and physical reactions are generally negative and/or straining, with damaging consequences, affecting negatively one or more of the health, capacities, performance, power, well-being, perceived happiness and/or live quality of a subject experiencing stress.

According to a definition, the term “stress” refers to or encompasses mental, emotional, psychic and/or physical strain, burden, exertion, or pressure due to overstraining and/or negative stimuli.

The term “stress” thus encompasses awareness or experiences of “stress in general”, a “stressful situation”, “acute stress”, “repeated exposure to stressful situations”, and/or “chronic stress”, for example.

In this regard, without wishing to be bound by theory, it is generally assumed that the present invention does not directly have the property of affecting the external or negative stimuli which may stress a subject. However, the composition of the invention is supposed to provide a feeling of well-being, in particular intestinal well being, which then positively affects a subject's (self-) awareness or in any other way reduces the feeling of stress and/or anxiety as perceived by the subject. The perception or feeling of stress and/or anxiety may thus in part be subjective or individual, with the composition of the invention preferably improving a subject's tenor, attitude, (intestinal) health, and/or well-being and thereby reducing stress and/or anxiety in the subject.

According to an embodiment, said stress is chronic stress. “Chronic stress” for the purpose of the present invention, is generally associated with the chronic, regular and/or repeated exposure to stressful situations. The stressful situations may be the same or may be different situations that occur at regular intervals or constantly, in particular so as to prevent occurrence of a less or non stressful, “normal” situation, in which a subject can rest, recover and/or experience recreation and/or regeneration. In this regard, chronic stress can also be seen as the prolonged absence of a possibility of rest, recovering, recreation and/or regeneration, in particular with respect to one or more specific external, negative stimulus.

“Chronic stress” generally lasts for at least one week or longer, for example 2, 3, 4, 5, weeks, in particular 1 month, 2 months 3 months and up to 6 months or even longer.

“Acute stress”, for the purpose of the present invention, is generally associated with exposure to one or a few punctual stressful situations, in particular occurring within a generally relatively short time interval, for example within a few minutes, one, two or six hours, possibly within one or two days (48 hours).

With respect to “acute stress”, the present invention preferably refers to the moment of the onset or beginning of a stressful state, for example at or shortly after the occurrence of one or several stressful situations, and thereby addresses in particular the intestinal symptoms and/or conditions that are associated with, related to, promoted by or the consequence of the onset of stress.

“Acute stress” also encompasses the situation where stress is (further) increased, for example in a subject already suffering from chronic stress.

The qualitative and quantitative assessment of the quality of life of an individual, and of whether or not a subject is stressed, and/or the severity or degree of stress experienced by a subject can be determined using psychological tests or medical questionnaires, such as those specified in the examples.

For example, the quality of life may be determined using QualityMetric's SF-36v2 Health survey.

The occurrence and the severity of stress may be determined (and thus also quantified) using, for example, Brantley P J, et al., Development and validation of the weekly stress inventory-short form. J. Psychopathol Behav Assess. 2007; 29:55-60.

Stress may also be determined and/or quantified using the Perceived Stress Scales (PSS), for example the original 14-item, a self-reported, unidimensional instrument to measure a perceived stress in response to situations in a person's life. Respondents report the prevalence of an item within the last month on a 5-point scale, ranging from never to very often (Cohen S, et al. A global measure of perceived stress. Journal of Health and Social Behavior, 1983; 24(4):385-396). A shorter, 10-item version (PSS 10-item questionnaire), which has been psychometrically tested (Cole 1999, J Epidemiol Community Health. 1999 May; 53(5): 319-320) may also be used.

According to an embodiment, the composition of the invention is suitable to reduce, treat and/or prevent anxiety. Anxiety may be determined, for example, using the Spielberger-State questionnaire (http://www.mindgarden.com/products/staisad.htm).

Of course, other tests, questionnaires or other scientific methodologies may also be used to determine the occurrence of stress and/or to quantify stress and/or anxiety, such as the degree, in particular the severity of stress and/or anxiety.

In an embodiment, the composition of the invention improves the quality of life of a subject consuming an effective amount of the composition by reducing stress, anxiety, by improving well-being in particular intestinal well-being, by relieving and addressing gastro-intestinal problems and symptoms as disclosed in this specification and by reducing the impact of stress on gastro-intestinal problems and symptoms.

According to an embodiment, the composition of the invention is suitable to address, relieve, reduce, prevent, and/or treat gastrointestinal symptoms and/or conditions.

The expressions “symptoms” and “conditions”, and their various grammatical forms, encompass but are not limited to the undesired and/or unpleasant intestinal situations mentioned in this specification. A “symptom” encompasses a perceived condition, which may but need not be a medical or a disease condition, such as the absence or impairment of well-being, happiness and/or high quality of life and/or the presence of a feeling of (gastrointestinal) sickness, physical and/or mental malaise, indisposition or illness.

The expressions “symptoms” and “conditions” may encompass digestive and/or gastrointestinal problems of any kind, and preferably those specified in this specification.

According to an embodiment, the composition of the invention is suitable to address, relieve, reduce, prevent, and/or treat lower intestinal symptoms and/or conditions.

According to an embodiment, the composition of the invention is suitable to address, relieve, reduce, prevent, and/or treat symptoms and/or conditions of the small intestine and/or the colon, preferably the colon.

According to an embodiment, the composition of the invention relieves, treats and/or prevents one or more selected from abdominal discomfort, abdominal pain, abdominal cramps, and bowel movement disturbances and/or irregularities

It is noted that the term “abdominal”, for example occurring in the expressions “abdominal discomfort”, “abdominal pain”, “abdominal bloating”, “abdominal distension”, “abdominal cramps”, generally and preferably refers to digestive, intestinal, bowel-related and/or gut-related symptoms and/or conditions.

Therefore, instead of the expression “abdominal discomfort”, the present invention preferably also envisages and encompasses “digestive discomfort”, “discomfort in a subject's digestive system”, “gut discomfort”, “bowel discomfort”, and “intestinal discomfort”, for example.

Similarly, the expression “abdominal pain” preferably also concerns and thus may be replaced, for the purpose of the present invention, with any one of the expressions “gut pain”, “digestive pain”, “pain in a subject's digestive system”, “bowel pain”, “intestinal pain”, for example.

Similarly, the expression “abdominal cramps” preferably also concerns and thus may be replaced with any one of the expressions “gut cramps”, “digestive cramps”, “cramps in or of a subject's digestive system”, “bowel cramps”, “intestinal cramps”, and the like.

Similarly, the expression “abdominal distension” preferably also concerns and thus may be replaced by any one of the expressions “gut distension”, “digestive distension”, “distension in or of a subject's digestive system”, “bowel distension”, “intestinal distension”, and the like.

Similarly, the expression “abdominal bloating” preferably also concerns and thus may be replaced by any one of the expressions “gut bloating”, “digestive bloating”, “bloating in or of a subject's digestive system”, “bowel bloating”, “intestinal bloating”, and the like.

The same applies in analogous manner to other intestinal symptoms and/or conditions disclosed in this specification. The composition of the invention may be used to relieve, reduce, treat and/or prevent any one of the symptoms and/or conditions specified in the above paragraphs and elsewhere in this specification.

The composition of the invention is shown to improve bowel habits, for example to improve the regularity of bowel movements.

The composition of the invention relieves, treats and/or prevents bowel transit disorders, such as those leading to constipation or diarrhea.

According to an embodiment, the composition of the invention is used for reducing the frequency and/or severity of intestinal symptoms, in particular any one or more of the intestinal symptoms disclosed elsewhere in this specification.

In particular, the composition of the invention is suitable to alleviate, reduce, prevent, and/or treat said gastrointestinal symptoms and/or conditions as specified in this specification in particular in as far as they are in any way related to stress.

For the purpose of the present specification, the term “related to”, for example in the context of the expression “related to stress”, refers to and is thus exchangeable with any one, several or all of the following terms: “in any way related to”, “associated with”, “promoted by”, “enhanced by”, “due to”, “the consequence of”, and “caused by”.

In other words, the present invention preferably addresses the link between stress and gastrointestinal symptoms and/or conditions.

According to an embodiment, the composition of the invention is used for relieving, treating and/or preventing one or more selected from stress-related abdominal discomfort, stress-related abdominal pain, stress-related abdominal cramps, and stress-related bowel movement disturbances and/or irregularities.

According to an embodiment, the composition of the invention relieves, treats and/or prevents stress-related bowel transit disorders, such as those leading to constipation or diarrhea.

According to an embodiment, the composition of the invention is used for restoring regular, normal and/or healthy bowel movements in subjects suffering from stress-related bowel movement disturbances and/or irregularities.

In an embodiment, the present invention provides the composition of the invention for regulating bowel movements in a subject suffering from and/or at a risk of stress-related bowel movement irregularities or disturbances, such as those specified in this specification.

In an embodiment, the present invention provides the composition of the invention for restoring the regularity and/or the normal, regular and/or healthy functioning of bowel movements in a subject suffering from stress-related bowel movement irregularities or disturbances, such as those specified in this specification.

In an embodiment, the present invention provides the composition of the invention for relieving, treating and/or preventing one or more selected from stress-related bloating, stress-related abdominal distension, stress-related flatulence, stress-related slow bowel transit, and stress-related constipation.

In an embodiment, the present invention provides the composition of the invention for relieving, treating and/or preventing stress-related rapid or accelerated bowel transit and/or stress-related diarrhea.

According to an embodiment, the composition of the invention is particularly useful for relieving, treating and/or preventing intestinal symptoms and/or conditions, in particular those disclosed in this specification, in as far as these symptoms and/or conditions are in any way related to chronic stress.

According to an embodiment, the composition of the invention is used for relieving, treating and/or preventing one or more selected from abdominal discomfort related to chronic stress, abdominal pain related to chronic stress, abdominal cramps related to chronic stress, and bowel movement disturbances and/or irregularities related to chronic stress.

According to an embodiment, the composition of the invention relieves, treats and/or prevents bowel transit disorders related to chronic stress, such as those leading to constipation or diarrhea.

According to an embodiment, the composition of the invention is used for restoring regular, normal and/or healthy bowel movements in subjects suffering from and/or at a risk of bowel movement disturbances and/or irregularities related to chronic stress.

In an embodiment, the present invention provides the composition of the invention for regulating bowel movements in a subject suffering from and/or at a risk of bowel movement irregularities or disturbances related to chronic stress, such as those specified in this specification.

In an embodiment, the present invention provides the composition of the invention for restoring the regularity and/or the normal, regular and/or healthy functioning of bowel movements in a subject suffering from and/or at a risk of bowel movement irregularities or disturbances, related to chronic stress such as those specified in this specification.

In an embodiment, the present invention provides the composition of the invention for relieving, treating and/or preventing one or more selected from abdominal bloating related to chronic stress, abdominal distension related to chronic stress, flatulence related to chronic stress, slow bowel transit related to chronic stress, and constipation related to chronic stress.

In an embodiment, the present invention provides the composition of the invention for relieving, treating and/or preventing rapid or increased intestinal transit related to chronic stress and/or diarrhea related to chronic stress.

According to another embodiment, the composition of the invention is particularly useful for relieving, treating and/or preventing intestinal symptoms and/or conditions as specified in this specification, which symptoms and/or conditions are in any way related to acute stress.

In particular, the present invention provides the composition of the invention for relieving, treating and/or preventing rapid or accelerated bowel transit related to acute stress and/or diarrhea related to acute stress.

According to an embodiment, the present invention is not or not specifically related to intestinal symptoms and/or conditions that are related to or the direct cause of any one or more selected from an infectious disease, a genetic disease, or an autoimmune disease, or any other specific disease.

In other words, in some embodiments, the present invention is suitable to address intestinal symptoms and/or conditions of generally healthy subjects and/or in the healthy population.

According to another, alternative embodiment, the present invention is used to relieve, treat and/or prevent intestinal symptoms and/or conditions associated any one of the aforementioned diseases or disease types, one or more selected from Irritable Bowel Syndrome (IBS), Inflammatory Bowel Disease (IBD), Ulcerative Colitis, Crohn's Disease, Microscopic Colitis, Organic gastrointestinal disease, such as Peptic Ulcer, Eosinophilic Gastroenteritis, Clostridium difficile or any bacterial infectious colitis, Celiac disease, gluten or lactose intolerance, for example. Preferably, the composition of the invention is useful to relieve, treat and/or prevent intestinal symptoms and/or conditions of subjects suffering from IBS.

Subjects suffering from certain disease such as those mentioned above may be and often are at risk of gastrointestinal problems as reported in this specification. The composition of the invention is thus particularly useful for such subjects. The composition is also advantageously used in case of other reasons and situations in which subjects suffer from and/or are exposed to an increased risk of getting one or more of the gastrointestinal conditions mentioned in this specification, which reasons and situations are known to the skilled person and are not further detailed here.

The composition of the invention is preferably intended to subjects. In particular, the benefits reported in this specification are preferably obtained in subjects. The composition is thus preferably served and/or administered to a subject. The subject preferably consumes the composition in order to achieve the benefits reported in his specification. Consumption and/or administration is, of course, preferably oral consumption, such as drinking, though other forms of enteral administration and/or nutrition, for example tubefeeding, such as nasogastric intubation, are not excluded from the scope of the present invention.

The subject may be a human or an animal. For example, the subject may be a pet or a livestock animal. For example, the animal may be selected from the group consisting of a pig, cattle (for example a cow, a sheep, a goat or a buffalo), a horse, poultry, a rabbit, a cat and a dog, and the like.

Preferably, subject is a human subjects and the composition of the invention is intended for human subjects. The human subject may be male or female. Furthermore, the human subject may be, for example, a child, a toddler, a teenager, an adult and/or an elderly person.

According to an embodiment, the composition of the invention is particularly advantageous for female subjects, for example women. Accordingly, said subject is preferably a woman.

According to another embodiment, the composition of the invention is particularly advantageous for male subjects, for example men.

According to an embodiment, the composition is destined to human subjects that have an age of 7 years, or less, up to 80 years, or more, for example for female subjects of this age.

According to an embodiment, the composition of the invention is particularly advantageous for human subjects that are 7-17 years old, for example for female subjects of this age.

According to an embodiment, the composition of the invention is particularly advantageous for human subjects that are that are 18-30 years old, for example for female subjects of this age.

According to an embodiment, the composition of the invention is particularly advantageous for human subjects that are 31-50 years old, for example for female subjects of this age.

According to an embodiment, the composition of the invention is particularly advantageous for human subjects that are 51-65 years old, for example for female subjects of this age.

According to an embodiment, the composition of the invention is particularly advantageous for human subjects that are older than 65 years, for example for female subjects of this age.

According to an embodiment, the composition of the invention is particularly advantageous for female human subjects, in particular of any one of the above age classes.

According to an embodiment, the composition of the invention is particularly useful for relieving, treating and/or preventing stress-related intestinal symptoms and/or conditions in women. Preferably, the composition is useful relieving, treating and/or preventing intestinal symptoms and/or conditions in female subjects, in particular in women, preferably in as far as such symptoms and/or conditions are related to chronic stress.

According to an embodiment, the composition of the invention is used for relieving, treating and/or preventing one or more selected from stress-related abdominal bloating, abdominal distension, flatulence, and constipation in women.

For example, the composition of the invention is used for relieving, treating and/or preventing one or more selected from abdominal discomfort related to chronic stress, abdominal pain related to chronic stress, abdominal cramps related to chronic stress, and bowel movement disturbances and/or irregularities related to chronic stress in a female subject, in particular in women.

According to an embodiment, the composition of the invention relieves, treats and/or prevents bowel transit disorders related to chronic stress, such as disorders leading to constipation or diarrhea, in a female subject, in particular a women.

In an embodiment, the present invention provides the composition of the invention for relieving, treating and/or preventing one or more selected from abdominal bloating related to chronic stress in women, abdominal distension related to chronic stress in women, flatulence related to chronic stress in women, slow bowel transit related to chronic stress in women, and constipation related to chronic stress women.

The present invention provides methods, such as a method for providing nutrition, a method for administrating the composition of the invention and/or methods for obtaining the various benefits reported in this specification.

The methods of the invention comprise the step of administering the composition of the invention. As mentioned above, the composition is preferably administered orally. As the composition is preferably provided in liquid or in powdered or otherwise reconstitutable form, the composition is preferably consumed by drinking.

Preferably, the composition is administered to a subject in need of the composition, for example to a subject suffering from stress, anxiety and/or the (intestinal) symptoms and/or conditions, as well as stress-related intestinal symptoms and/or conditions specified in this specification. By consuming the composition of the invention and/or by having the composition administered, in particular as specified in this specification, the beneficial effects are obtained.

Preferably, an effective amount of the composition is administered. In this regard, the skilled person may determine the amount that is effective for any particular subject, for example any specific animal. Preferably, the serving sizes and/or daily servings as specified elsewhere in this specification are administered.

Preferably, the composition of the invention is administered over more than one day, preferably for 2, 3, 4, 5, 6, 7, or more days, preferably for 1 week or longer, 2, 3, 4, 5, or 6 weeks or longer, preferably 1 month, 2, 3, 4, 5 months or longer. The composition of the invention does preferably not have any adverse or side effect on the normal, average consumer, and may thus be consumed over longer time periods, for example for months or years, thereby exerting its beneficial effects.

Without wishing to be bound by theory, it is supposed that the beneficial and surprising effects reported in this specification are in particular achieved through the synergistic combination of the components and ingredients of the composition of the invention, comprising in particular soluble fiber and one or more probiotic and/or medium fermented by a probiotic, in particular in accordance with aspects and preferred embodiments detailed in this specification, besides possibly further ingredients and components.

### EXAMPLES

The following examples are illustrative of some of the products and methods of making the same falling within the scope of the present invention. They are not to be considered in any way limitative of the invention. Changes and modifications can be made with respect to the invention. The skilled person will recognize many variations in these examples to cover a wide area of formulas, ingredients, processing and mixtures to rationally adjust the nutrients and other elements of the invention for a variety of applications.

### Example 1

### A Powdered Composition Comprising Oligo-Glucosaccharide Fiber and a Probiotic Culture Powder

A powdered composition is produced by preparing an aqueous slurry of ingredients, in particular skimmed milk powder, corn syrup, oligo-glucosaccharide fiber (Fibersol-2®, commercially obtained from Matsutani America, Inc., USA), sweet whey, maltodextrin, milk fat, flavouring ingredients, an emulsifier, and a vitamin mix, stabilizers and thickeners as necessary. The slurry is subjected to heating for evaporation and then transformed to a powder by spray-drying.

Thereafter, about 1.4 g of a probiotic culture powder, containing spray-dried Lactobacillus paracasei, deposited at the “Collection Nationale de Cultures de Microorganismes” (CNCM) under numbers CNCM 1-2116 and CNCM 1-1292 culture powder is added so as to provide at least 0.5-2×10⁷ CFU per 1 ml of reconstituted composition (live probiotic).

The nutrient composition per 100 grams of dry matter of the composition is provided in Table 1 below. Residual moisture is not shown in Tables 1-3, but is generally 6% of the weight of the composition (including water), or less, preferably 5% or less.

The composition of Table 1 contains about 330 kcal (1380 kJoules) per 100 g dry matter.

TABLE 1
Dry matter composition of composition 1 according
to an embodiment of the invention (probiotic
added in the form of a culture powder).

| Nutrient | Unity | weight per 100 g dry matter |
|---|---|---|
| Fat | g | 1.78 |
| Milk fat | g | 1.46 |
| Non-milk Fat | g | 0.10 |
| Lecithin | g | 0.22 |
| Protein | g | 21.07 |
| Casein | g | 15.43 |
| Whey protein | | 5.03 |
| Other | g | 0.62 |
| Available carbohydrates | g | 59.70 |
| Lactose | g | 35.88 |
| Maltodextrin | g | 22.45 |
| Other (mainly sugars and | g | 1.38 |
| digestible oligosaccharides) | | |
| Dietary fiber | g | 9.43 |
| Oligo-glucosaccharides | g | 9.43 |
| (Fibersol-2 ®) | | |
| Minerals (ash) | g | 8.01 |
| Sodium | mg | 298.52 |
| Potassium | mg | 1099.82 |
| Chloride | mg | 759.40 |
| Calcium | mg | 1885.41 |
| Phosphorus | mg | 635.80 |
| Magnesium | mg | 76.46 |
| Manganese | μg | 329.95 |
| Selenium | μg | 6.60 |
| Micronutrients | mg | 145.89 |
| Vitamin A (retinol) | μg | 440.00 |
| | RE¹ | |
| Vitamin D | μg | 6.00 |
| | CE² | |
| Vitamin E | mg | 18.00 |
| | TE³ | |
| Vitamin K | μg | 0.50 |
| Vitamin B₂ | mg | 1.40 |
| Vitamin C | mg | 90.00 |
| Thiamin (B₁) | mg | 0.40 |
| Niacin (vit B₃) | mg | 6.50 |
| Pyridoxin (vit B₆) | mg | 0.65 |
| Biotin (B₈) | μg | 50.00 |
| Folic acid (vit B₉) | μg | 480.00 |
| Pantothenic acid | mg | 1.85 |
| Cobalamin (vit B₁₂) | μg | 1.80 |
| Iron | mg | 17.00 |
| Zinc | mg | 2.50 |
| Total | g | 100.00 |

¹RE = Retinol Equivalents
²CE = Cholecalciferol equivalents
³TE = alpha-tocopherol equivalents

### Example 2

### Nutritional Composition Comprising Soluble Fiber and a Fermented Milk Powder

A powdered composition is produced as described for Example 1 above, with the difference that a fermented milk powder of the same Lactobacillus paracasei strain instead of a culture powder was used, to also provide at least 0.5-2×10⁷ CFU per 1 ml of reconstituted composition (live probiotic). The nutrient composition of this example is shown in Table 2 below, in which micronutrients (vitamins, trace elements) are present in substantially the same amounts as in Table 1 above, and are thus not shown anew. The composition has about the same energy content as the composition of Example 1.

TABLE 2
Dry matter composition of composition 2 according
to an embodiment of the invention (probiotic
added in the form of fermented milk powder).

| Nutrient | Unity | weight per 100 g dry matter |
|---|---|---|
| Fat | g | 1.66 |
| Milk fat | g | 1.29 |
| Non-milk Fat | g | 0.16 |
| Lecithin | g | 0.22 |
| Protein | g | 22.68 |
| Casein | g | 16.66 |
| Whey protein | | 5.40 |
| Other | g | 0.61 |
| Available carbohydrates | g | 58.60 |
| Lactose | g | 34.35 |
| Maltodextrin | g | 22.84 |
| Other (mainly sugars and | g | 1.41 |
| digestible oligosaccharides) | | |
| Dietary fiber | g | 9.36 |
| Oligo-glucosaccharides | g | 9.36 |
| (Fibersol-2 ®) | | |
| Minerals (ash) | g | 7.70 |
| Sodium | mg | 296.44 |
| Potassium | mg | 1092.16 |
| Chloride | mg | 754.11 |
| Calcium | mg | 1872.27 |
| Phosphorus | mg | 683.38 |
| Magnesium | mg | 78.01 |
| Manganese | μg | 248.60 |
| Selenium | μg | 6.86 |
| Total | g | 100.00 |

### Example 3

### Nutritional Composition Comprising Soluble Fiber and Heat-Inactivated Probiotics and their Fermented Medium

A powdered composition is produced as described for Examples 1 and 2 above, with the difference that a mix of lyophilized, heat-inactivated lactobacilli (L. fermentum and L. delbrueckii) in the form of the commercially available product Lacteol® (www.pohl-boskamp.com) was added instead of a culture powder comprising live probiotic.

The concentration of L. fermentum and L. delbrueckii, before inactivation, was 0.25-1×10⁷ CFU each per 1 ml of reconstituted composition, so as to correspond to the same total amount of probiotics used in Examples 1 and 2. The nutrient composition of this example is shown in Table 3 below, in which micronutrients (vitamins, trace elements) are present in substantially the same amounts as in Table 1 above, and are thus not shown anew.

The composition of Table 3 has substantially the same energy content as the compositions of Examples 1 and 2 above.

TABLE 3
Dry matter composition of composition 1 according to an embodiment
of the invention (probiotic added in the form of heat inactivated
mixture of two lactobacilli and their fermented medium).

| Nutrient | Unity | weight per 100 g dry matter |
|---|---|---|
| Fat | g | 1.72 |
| Milk fat | g | 1.45 |
| Non-milk Fat | g | 0.06 |
| Lecithin | g | 0.21 |
| Protein | g | 20.81 |
| Casein | g | 15.59 |
| Whey protein | | 5.13 |
| Other | g | 0.09 |
| Available carbohydrates | g | 60.66 |
| Lactose | g | 35.70 |
| Maltodextrin | g | 23.67 |
| Other (mainly sugars and | g | 1.30 |
| digestible oligosaccharides) | | |
| Dietary fiber | g | 9.37 |
| Oligo-glucosaccharides | g | 9.37 |
| (Fibersol-2 ®) | | |
| Minerals (ash) | g | 7.44 |
| Sodium | mg | 296.60 |
| Potassium | mg | 1092.73 |
| Chloride | mg | 754.50 |
| Calcium | mg | 1873.24 |
| Phosphorus | mg | 683.73 |
| Magnesium | mg | 78.05 |
| Manganese | μg | 248.73 |
| Selenium | μg | 6.87 |
| Total | g | 100.00 |

### Example 4

### A Clinical Trial to Assess Impact of the Compositions of the Invention on Stress-Related Gastrointestinal Symptoms and/or Conditions

A double blind, placebo-controlled, exploratory, randomized, and parallel design clinical trial of four groups is performed.

### 1. Objectives of the Trial

The objective of the trial is to assess the effect of the composition of the invention on stress-related lower intestinal symptoms. Such symptoms or conditions include frequency and/or severity of abdominal discomfort, pain and/or cramps; bowel movement disturbances (constipation and diarrhea, as defined by the assessment of stool consistency and frequency).

Secondary objectives of the trial are the assessment of the capacity of the composition of the invention to reduce abdominal bloating/distension and/or flatulence (frequency and severity); to improve overall intestinal well-being; to reduce the impact of stress on intestinal symptoms; to improve the quality of life; to reduce stress; to reduce anxiety.

It is also assessed if any outcome is gender and/or age specific.

### 2. Trial Design, Subjects, Groups and Duration

This study is addressed to 18-65 year-old males and females of any ethnicity self-reporting frequent lower intestinal symptoms and showing a perceived stress score ≧14 (see below).

In each study group, 38 subjects are enrolled, and new subjects are recruited in case the drop-out rate is higher than 20% to insure that at least 30 subjects in each group complete the protocol.

The trial has one placebo control group (Group 1) and three experimental treatment groups. Subjects are assigned to one of the following treatment groups:

Group 1: receiving a skimmed milk powder (placebo)
Group 2: the nutritional composition of Example 1.
Group 3: the nutritional composition of Example 2.
Group 4: the nutritional composition of Example 3.

Comparisons are run between the experimental groups vs. placebo control group.

Subjects meeting the pre-inclusion criteria go through a 2 weeks run-in period. Only those fulfilling all inclusion criteria are eligible to continue the study and begin the treatment period that lasts 5 weeks (total duration is thus 7 weeks).

### 3. Study Population, Inclusion and Exclusion Criteria

Males or females aged 18-65 years on the day of the pre-inclusion visit (V0), reporting at least one of the following lower intestinal symptoms: abdominal discomfort/pain/cramps and/or the more specific abdominal bloating/distension, or flatulence/passage of gas with, OR having disturbed bowel movements for more than half of the time of the run-in period (period between V0 and V1, see below) 1), AND showing a perceived stress score ≧14 (Perceived Stress Scale PSS-10 questionnaire, see below) are included in the study. Only females under effective contraception (oral contraception, chirurgical sterilization, coil) are included. Furthermore, individuals need to have provided his/her informed consent and need to be willing to comply with the study procedures.

The perceived stress score is determined on the basis of Perceived Stress Scale (PSS), which was originally developed as a 14-item, self-reported, unidimensional instrument to measure a perceived stress in response to situations in a person's life. Respondents report the prevalence of an item within the last month on a 5-point scale, ranging from never to very often (Cohen S, et al. A global measure of perceived stress. Journal of Health and Social Behavior, 1983; 24(4):385-396). For the present study, the 10-item version (PSS 10-item questionnaire), which has been psychometrically tested (Cole 1999, J Epidemiol Community Health. 1999 May; 53(5): 319-320) is used.

Individuals meeting at least one of the following criteria are excluded from the study: pregnant or lactating women; diagnosis of Inflammatory Bowel Disease (Ulcerative Colitis, Crohn's Disease, Microscopic Colitis), Organic gastrointestinal disease (such as Peptic Ulcer, Eosinophilic Gastroenteritis, Clostridium difficile or any bacterial infectious colitis), Celiac disease, gluten or lactose intolerance, known allergy to cow-milk proteins, under prescription for medication for intestinal symptoms (laxatives, anti-diarrhoeals, antiacids, antirreflux, medicines, proton pump inhibitors, antispasmodics, prokinetics, any other probiotics, fibre supplements, etc), digestive tract surgery except appendectomy, haemorrhoidectomy and cholecystectomy, systemic disease with pain that may interfere with the self assessment of abdominal pain, currently receiving antibiotic treatment, or having taken such a treatment in the last week before pre-inclusion (V0), currently suffering from, or having suffered from, major psychiatric or neurotic disorder/pathology within the past year prior to the inclusion visit (V0), currently receiving medical treatment of stress-induced symptoms (such as sleeping pills, antidepressants, anxiolytics, tranquilizers), or having taken such a treatment in the last 3 months, alcohol abuse, drug addiction, currently participating or having participated in another clinical trial during the last 3 months prior to the beginning of this study, having holidays during the treatment phase.

During the whole trial, subjects are asked to exclude from their diet any kind of fermented food or drink/probiotic treatment, with the exception of the dairy product tested.

### 4. Treatments, Compliance During the Study, Product Handling

For each group, feeding with the assigned study product begins on Day 1 after inclusion, and then continues until Day 35 (5 weeks duration).

All milk-based drinks tested (groups 1-4) are provided in a powder format, packed in stickpacks, and are stable at room temperature (25° C.), ready to be dissolved in water for oral consumption.

Each stickpack contains 24 g of powder to be dissolved with 200 mL of water, to yield a 250 mL milk drink. For each formulation, 2 servings of 250 mL per day are taken by the subjects during the entire study period. The first drink is taken with breakfast and the second one with diner or before going to bed. The formulations deliver about 35 kcal per 100 mL of the drinks, about 5 g of soluble oligo-glucosaccharide fiber per day (groups 2, 3 and 4), and at least 1E+09 CFU of L. paracasei (groups 2 and 3) or of live equivalent L. fermentum+L. delbrueckii (group 4) per day.

All formulations are labeled with codes, study number, expiry date, and storage conditions (room temperature).

Compliance with the protocols is assessed during the study. Unauthorized diets, treatments or medications during the study period include the medication already mentioned with respect to exclusion criteria (no. 3. above), furthermore prolonged use of analgesics, and consumption of any type of yoghurts, fermented milk based drinks, or probiotics containing products (any commercially available product specified as containing Lactobacillus, Bifidobacteria, Streptococcus, Saccharomyces fifteen days before the day of the pre-inclusion visit (V0) and up to the end of the 5 weeks treatment phase.

### 5. Measures and Assessments

In order to meet the primary objectives of the study, the frequency and severity of abdominal discomfort, pain and/or cramps; stool consistency; and stool frequency were measured after 5 weeks of treatment using an adapted form of the methodology disclosed in Guyonnet D. et al. Fermented milk containing Bifidobacterium lactis DN-173 010 improved self-reported digestive comfort amongst a general population of adults. A randomized, open-label, controlled, pilot study. J Dig Dis. 2009; 10(1):61-70):

The questionnaires to be filled in by the subjects contain the questions as follows:

1) Frequency of abdominal discomfort/pain/cramps after 5 weeks of treatment: Over the past week, I have been bothered by abdominal discomfort/pain/cramps: 0—Never; 1—No more than once a week; 2—2-3 days a week; 3—4-5 days a week; 4—Every day of the week.

2) Severity of abdominal discomfort/pain/cramps after 5 weeks of treatment, as follow: Over the past week, my abdominal discomfort/pain/cramps symptoms were: 0—Non-existent; 1—Mild; 2—Moderate; 3—Severe; 4—Very severe.

3) Stool consistency after 5 weeks of treatment is assesses according to the Bristol stool form scale, which is filed-in on a daily basis for each bowel movement (Lewis S. J et al. Stool form scale as a useful guide to intestinal transit time. Scand J Gastroenterol. 1997; 32:920-924). See also http://en.wikipedia.org/wiki/Bristol_Stool_Scale.

The daily recorded points are averaged over the last week of treatment. Baseline are mean scores at 2^{nd} week of the run-in period.

4) Number of bowel movements a day after 5 weeks of treatment are assessed daily, in a diary, as follows: Today, how many bowel movements did you have? 0 time; 1 time; 2 time; 3 times; 4 times; 5 times or more.

The daily recorded points are averaged over the last week of treatment. Baseline are mean scores at 2^{nd} week of the run-in period.

To assess further objectives of the trial (the secondary objectives), the following measures or assessments are collected:

The frequency of abdominal discomfort/pain/cramps is determined after 2, 3, and 4 weeks of treatment as described above (no. 1)).

The severity of abdominal discomfort/pain/cramps is determined after 2, 3, and 4 weeks of treatment as described above (no. 2)).

The consistency of stools is determined after 2, 3, and 4 weeks of treatment as described above (no. 3)).

The number of bowel movements a day after 2, 3, and 4 weeks of treatment is determined as detailed above (no. 4)).

Further measures or assessments are made using questionnaires:

5) The frequency of abdominal bloating/distension is determined after 2, 3, 4 and 5 weeks of treatment using the following question (adapted from (Guyonnet et al. (2009)): Over the past week, I have been bothered by abdominal bloating/distension: (0) Never; (1) No more than once a week; (2) 2-3 days a week; (3) 4-5 days a week; (4) Every day of the week.

6) The frequency of flatulence/passage of gas is determined after 2, 3, 4 and 5 weeks of treatment with the following question: Over the past week, I have been bothered by flatulence/passage of gas. The reply options are the same from never to every day as under 5) above.

7) The severity of abdominal bloating/distension symptoms is determined after 2, 3, 4 and 5 weeks of treatment with the following question: Over the past week, my abdominal bloating/distension symptoms were: (0) non-existent; (1) Mild; (2) Moderate; (3) Severe; (4) Very severe.

8) The severity of flatulence/passage of gas symptoms is determined after 2, 3, 4 and 5 weeks of treatment with the following question: Over the past week, my flatulence/passage of gas symptoms were. The reply options are the same from non-existent to very severe as under 7) above.

9) The overall intestinal well-being is determined after 2, 3, 4 and 5 weeks of treatment as with the following question: How do you consider, in the past seven days, your intestinal well being (stool frequency and consistency, abdominal discomfort/pain/cramps, abdominal bloating/distension, flatulence/passage of gas) compared to the period before beginning the consumption of the study product? The reply options are: Worsened; No change; Improved.

10) The impact of stress on intestinal symptoms is determined after 2, 3, 4 and 5 weeks of treatment using the questions a)-c) below, with the following reply options: (0) Totally disagree; (1) Mostly disagree; (2) Don't know; (3) Mostly agree; (4) Totally agree.

a) I believe that any stress causes my digestive problems.
b) Major aggravation triggers my digestive problems.
c) Even the least bit of aggravation triggers my digestive problems.

11) Quality of life of subjects is determined after 5 weeks of treatment using the QualityMetric's SF-36v2 Health survey (http://www.qualitymetric.com).

12) Stress is determined after 2, 3, 4 and 5 weeks of treatment using the weekly stress inventory—short form according to Brantley P J, Bodenlos J S, Cowles M, Whitehead D, Ancona M, Jones G N. Development and validation of the weekly stress inventory-short form. J. Psychopathol Behav Assess. 2007; 29:55-60.

13) Stress is determined after 5 weeks of treatment using the Perceived Stress Scale (PSS-10) described above (no 3.) (Cohen et al. 1983) and compared to baseline (inclusion visit) values.

14) Stress is further determined after 5 weeks of treatment by measuring blood pressure and by measuring saliva cortisol level (Pruessner M. et al. Psychosom Med. 2003; 65(1):92-9) and compared to baseline (inclusion visit) values.

15) Anxiety is determined after 5 weeks of treatment using the Spielberger-State questionnaire (http://www.mindgarden.com/products/staisad.htm).

### 6. Conduct of the Trial

Subjects fulfilling pre-inclusion criteria (i.e. stress and lower intestinal symptoms, see no. 3. above), presenting none of the exclusion criteria, and signing the informed consent are enrolled at a hospital. Their bowel habits (stool frequency and consistency) are followed daily during a 2 weeks run-in period, and the frequency of their lower intestinal symptoms is recorded weekly. Based on these recorded data, subjects confirmed to match with our inclusion criteria (stress and lower intestinal symptoms) are included in the treatment phase of the clinical trial. They are randomized into 4 groups to receive one of the nutritional formulations (see no. 2. above) that they will consume daily during the next 5 weeks.

The frequency and severity of lower intestinal symptoms, bowel habits, the impact of stress on digestive problems as perceived by subjects, the overall intestinal well-being, and the stress are scored using questionnaires after 2, 3, 4 and 5 weeks of dietary treatment. The anxiety and the quality of life are assessed after 5 weeks of treatment. Finally, the stress level is also assessed after 5 weeks of treatment by measuring cortisol levels in saliva and blood pressure.

### 7. Statistics

Subjects are assigned to treatment groups by stratified randomization. The strata are: gender (female, male); age (18-30, 30-50, 50-65); abdominal discomfort/pain/cramps severity at baseline (mild, moderate, severe, very severe); abdominal discomfort/pain/cramps frequency at baseline (no more than once a week, 2-3 days a week, 4-5 days a week, every day of the week). Effects to be estimated are the treatment differences of the three experimental groups with respect to the placebo control group.

### 8. Other Aspects

The health of all subjects is monitored throughout the study.

The trial is conducted in accordance with relevant legal requirements and is only started once written approval of the Independent Ethics Committee (ICE) and Health authorities are received. The trial is conducted according to the principles and rules laid down in the Declaration of Helsinki and its subsequent amendments.

Furthermore, this clinical trial is conducted following the principles of ICH (International Conference on Harmonization) guideline for Good Clinical Practice and adherence with the applicable regulatory or legal requirements.

### 9. Results

The results show that the composition of the invention is suitable to reduce intestinal conditions, in particular lower intestinal condition.

In particular, the composition reduces the frequency of abdominal discomfort, pain and/or cramps, compared the control compositions.

The composition reduces the severity of intestinal symptoms such as abdominal discomfort, pain and/or cramps, compared a control composition.

Furthermore, the composition of the invention reduces the frequency and severity of intestinal symptoms and/or conditions.

The composition of the invention is shown to improve the overall intestinal well-being.

The composition of the invention is shown to improve bowel habits, that is, to improve the regularity of bowel movements, by improving transit disorders leading to constipation or diarrhea.

The composition of the invention is shown to reduce diarrhea.

The composition of the invention is shown to reduce constipation, bloating, distension and/or flatulence.

The composition of the invention is shown to reduce stress. The composition of the invention is shown to reduce anxiety.

The composition of the invention is shown to improve the quality of life.

In particular, the composition of the invention is shown to reduce the above conditions and symptoms in as far as they are stress-related, caused by stress and/or aggravated by stress.

The composition reduces the impact or effect of stress on intestinal conditions and symptoms mentioned in this specification.

The composition of the invention is shown to reduce, in particular, stress related intestinal symptoms occurring due to chronic stress.

The composition of the invention, in particular the composition comprising live probiotic, is shown to restore regular digestion following stress-related bowel movement disturbances, in particular due to chronic and/or acute stress. In this regard, the composition is shown to reduce constipation, bloating, distension, flatulence related to chronic stress, in particular for women and/or men.

The composition of the invention, in particular the composition comprising inactive probiotic, is shown to restore bowel regularity also in patients suffering from diarrhea related to stress, for example acute stress.

The composition of the invention is particularly effective for reducing intestinal symptoms and/or conditions as specified in herein in women and/or men, in as far as these conditions are related to chronic stress.

Interestingly, the composition comprising a mixture of different probiotic strains, for example different lactobacilli strains, is suitable to improve bowel regularity, in particular bowel movement regularity or to reduce stress-related bowel movement disturbances, also in patients suffering from diarrhea.

1. A composition comprising at least one soluble fiber and at least one selected from (a) a live probiotic, (b) an inactive probiotic, (c) a culture medium of a probiotic and (d) a combination of two or more of (a), (b) and (c), for relieving, treating and/or preventing intestinal symptoms and/or conditions related to chronic stress.

2. The composition of claim 1, wherein said at least one soluble fiber comprises or substantially consists of low-viscosity soluble fiber.

3. The composition of any one of the preceding claims, wherein said at least one soluble fiber comprises or substantially consists of soluble oligosaccharide fiber.

4. The composition of any one of the preceding claims, wherein said intestinal symptoms and/or conditions are selected from one or more of abdominal discomfort, abdominal pain, abdominal cramps, and bowel movement disturbances and/or irregularities.

5. The composition of any one of the preceding claims, wherein said intestinal symptoms and/or conditions are selected from one or more of abdominal bloating, abdominal distension, flatulence, slow bowel transit and constipation.

6. The composition of any one of the preceding claims, wherein said intestinal symptom and/or condition related to stress is increased intestinal transit and/or diarrhea.

7. The composition of any one of the preceding claims, wherein said chronic stress is associated with the chronic, regular and/or repeated exposure to stressful situations.

8. The composition of any one of the preceding claims, for relieving, treating and/or preventing intestinal symptoms and/or conditions related to chronic stress in women.

9. The composition of any one of the preceding claims, for relieving, treating and/or preventing one or more selected from abdominal discomfort, abdominal pain, abdominal cramps, and bowel movement disturbances related to chronic stress in women.

10. The composition of any one of the preceding claims, for relieving, treating and/or preventing one or more selected from abdominal bloating, abdominal distension, flatulence, slow bowel transit and constipation related to chronic stress in women.

11. A composition comprising at least one soluble fiber and at least one selected from (a) a live probiotic, (b) an inactive probiotic, (c) a culture medium of a probiotic and (d) a combination of two or more of (a), (b) and (c), for restoring regular, normal and/or healthy bowel movement in a subject suffering from bowel movement irregularities related to stress.

12. The composition of claim 11, wherein said stress is chronic stress.

13. The composition of any one of claims 11 and 12, wherein said subject is a female subject, in particular a woman.

14. A method for relieving, treating and/or preventing intestinal symptoms and/or conditions related to chronic stress, the method comprising administering to a subject in need thereof an effective amount of a composition comprising at least one soluble fiber and at least one selected from (a) a live probiotic, (b) an inactive probiotic, (c) a culture medium of a probiotic and (d) a combination of two or more of (a), (b) and (c).

15. The method of claim 14, wherein said intestinal symptom and/or condition is selected from one or more of abdominal discomfort, abdominal pain, abdominal cramps, and bowel movement disturbances and/or irregularities.
